(19) United States
(12) Patent Application Publication
(10) Pub. No.: US 2023/0384287 A1
(43) Pub. Date: Nov. 30, 2023

(54) ANALYSIS APPARATUS, ANALYSIS METHOD, AND STORAGE MEDIUM

(71) Applicant: CANON MEDICAL SYSTEMS CORPORATION, Otawara-shi (JP)

(72) Inventor: Shozo HASHIMOTO, Nasushiobara (JP)

(73) Assignee: CANON MEDICAL SYSTEMS CORPORATION, Otawara-shi (JP)

(21) Appl. No.: 18/322,017

(22) Filed: May 23, 2023

(30) Foreign Application Priority Data
May 24, 2022 (JP) 2022-084530
Jun. 7, 2022 (JP) 2022-092340
May 9, 2023 (JP) 2023-077387

(51) Int. Cl. G01N 33/49; A61B 5/15; G16H 40/63

(52) U.S. Cl. CPC G01N 33/49; A61B 5/150022; G16H 40/63

(57) ABSTRACT

An analysis apparatus of embodiments includes a holder and processing circuitry, and the holder holds a blood sample collected from a subject. The processing circuitry measures at least one of the viscoelasticity or the viscosity of the held blood sample via a mediator in contact with the held blood sample.

### CROSS-REFERENCE TO RELATED APPLICATION

The present application claims priority based on Japanese Patent Application No. 2022-084530 filed May 24, 2022, Japanese Patent Application No. 2022-092340 filed Jun. 7, 2022, and Japanese Patent Application No. 2023-077387 filed May 9, 2023, the contents of which are incorporated herein by reference.

### FIELD

Embodiments disclosed in the specification and drawings relate to an analysis apparatus, an analysis method, and a storage medium.

### BACKGROUND

There are measuring devices that analyze the blood of the human body and measure the viscoelasticity of the blood. Conventional measuring devices cannot perform real-time measurement, and thus it takes a long time to obtain results of measuring viscoelasticity after samples are placed on the measuring devices. Further, used samples are discarded and consequently consumed.

### BRIEF DESCRIPTION OF THE DRAWINGS

FIG. 1 is a diagram showing an example of a configuration of an analysis apparatus 100 of an embodiment.

FIG. 2 is an explanatory diagram showing an overview of a measuring device 120.

FIG. 3 is a diagram showing an example of a measuring device 1 of a first embodiment.

FIG. 4 is a diagram showing the operation of the measuring device 1 of the first embodiment at the time of calculating the viscoelasticity of a sample LB.

FIG. 5 is a diagram showing the operation of a measuring device 1 of a second embodiment at the time of calculating the viscoelasticity of the sample LB.

FIG. 6 is a diagram showing an example of a measuring device 1 of a third embodiment.

FIG. 7 is a diagram showing a modified example of the measuring device 1 of the third embodiment.

FIG. 8 is a block diagram showing an analysis system of a fourth embodiment.

FIG. 9 is a flowchart showing analysis processing of the analysis system of the fourth embodiment.

FIG. 10 is a diagram showing a first structural example of a channel device according to the fourth embodiment.

FIG. 11 is a diagram showing a first process of a viscoelasticity measurement method according to the first structural example.

FIG. 12 is a diagram showing a second process of the viscoelasticity measurement method according to the first structural example.

FIG. 13 is a diagram showing a third process of the viscoelasticity measurement method according to the first structural example.

FIG. 14 is a diagram showing a second structural example of the flow channel device according to the fourth embodiment.

FIG. 15 is a diagram showing a first process of a viscoelasticity measurement method according to the second structural example.

FIG. 16 is a diagram showing a second process of the viscoelasticity measurement method according to the second structural example.

FIG. 17 is a diagram showing a third process of the viscoelasticity measurement method according to the second structural example.

FIG. 18 is a diagram showing a modified example of the second structural example.

FIG. 19 is a conceptual diagram showing a usage example of the analysis system according to the fourth embodiment.

FIG. 20 is a conceptual diagram showing a usage example of an analysis system according to a fifth embodiment.

FIG. 21 shows a configuration example of a flow channel device according to the fifth embodiment.

### DETAILED DESCRIPTION

Hereinafter, an analysis apparatus, an analysis method, and a storage medium of embodiments will be described with reference to the drawings.

The analysis apparatus of embodiments includes a holder and processing circuitry, and the holder holds a blood sample collected from a subject. The processing circuitry measures at least one of the viscoelasticity or the viscosity of the held blood sample via a mediator in contact with the held blood sample.

### First Embodiment

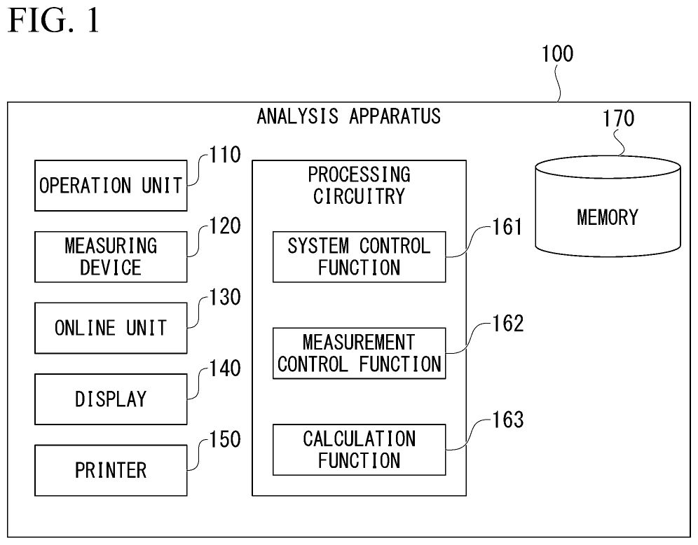

FIG. 1 is a diagram showing an example of a configuration of an analysis apparatus 100 of an embodiment. The analysis apparatus 100 includes, for example, an operation unit 110, a measuring device 120, an online unit 130, a display 140, a printer 150, processing circuitry 160, and a memory 170. The analysis apparatus 100 calculates the viscoelasticity of human blood (hereinafter referred to as a sample, a blood sample) on the basis of a detection result of the measuring device 120.

The operation unit 110 includes an input interface such as a keyboard, a mouse, buttons, and a touch key panel, for example. The input interface in this specification is not limited to those having physical operation components such as a mouse and a keyboard. For example, examples of the input interface also include electrical signal processing circuitry that receives an electrical signal corresponding to an input operation from an external input device provided separately from the apparatus and outputs the electrical signal to a control circuit.

Various operations are performed in the operation unit 110. Operations performed by the operation unit 110 include, for example, setting of analysis conditions, input of subject information such as a subject ID and a subject name, selection of measurement items for each test sample of a subject, calibration operation for each item, test sample analysis operation, and the like.

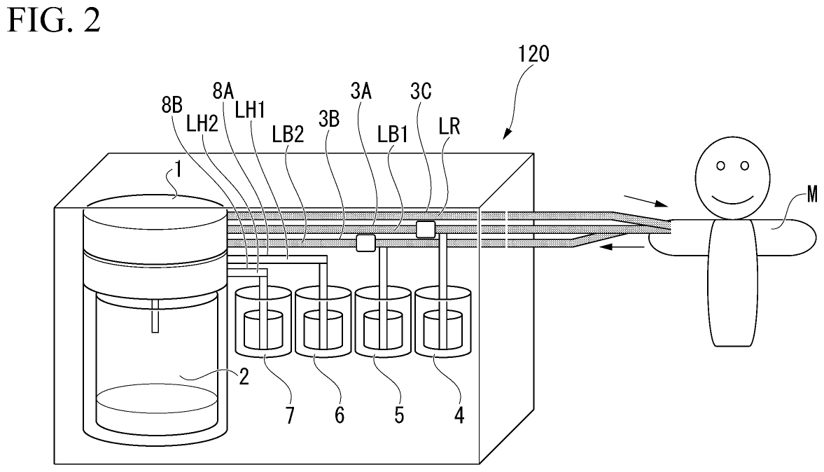

The measuring device 120 detects data for calculating the viscoelasticity of a human body sample (hereinafter referred to as calculation element data). FIG. 2 is an explanatory diagram showing an overview of the measuring device 120. The measuring device 120 includes, for example, a measuring device 1, a waste bottle 2, and a tube 3. The tube 3 can communicate with the blood vessel of a subject M via, for example, a syringe that is not shown, and thus some sample in the blood vessel is supplied into a first tube 3A and a second tube 3B. The sample supplied to the first tube 3A and the second tube 3B is transferred to the measuring device 1. The sample used for measurement may be discarded or may be returned to the subject M through circulation via a return tube 3C.

A mixing area for mixing an anticoagulant and the sample is provided in the middle of the first tube 3A and the second tube 3B. A first anticoagulant container 4 is connected to the first tube 3A and a second anticoagulant container 5 is connected to the second tube 3B. The first anticoagulant container 4 contains a low-concentration anticoagulant. The second anticoagulant container 5 contains a high-concentration anticoagulant.

A sample mixed with the low-concentration anticoagulant (hereinafter referred to as a first sample) LB1 flows through the first tube 3A. A sample mixed with the high-concentration anticoagulant (hereinafter referred to as a second sample) LB2 flows through the second tube 3B. In the following description, when the first sample LB1 and the second sample LB2 are not distinguished from each other, they are collectively referred to as a sample LB. The anticoagulant is prepared in a plurality of different types of concentrations, from low concentrations to high concentrations. The anticoagulant may be prepared in a greater variety of concentrations.

The effect of the anticoagulant on the viscoelasticity of the sample LB may be measured by measuring the viscoelasticity of two samples LB mixed with the anticoagulant in different concentrations. This makes it possible to obtain the amount of anticoagulant that makes the samples LB have a predetermined viscoelasticity (coagulability). An area for mixing physiological saline may be provided in the middle of the tube 3. Clogging of a channel may be prevented by adding a predetermined amount of anticoagulant to the sample LB conveyed through the tube 3. If an anticoagulant is added, the analysis apparatus 100 may report the amount of anticoagulant added to a user.

A first standard sample container 6 and a second standard sample container 7 are further connected to the measuring device 1 via a third tube 8A and a fourth tube 8B, respectively. The first standard sample container 6 contains a standard sample mixed with a low-concentration anticoagulant (hereinafter referred to as a first standard sample LH1). The second standard sample container 7 contains a standard sample mixed with a high-concentration anticoagulant (hereinafter referred to as a second standard sample LH2). In the following description, when the first standard sample LH1 and the second standard sample LH2 are not distinguished from each other, they are collectively referred to as a standard sample LH.

The measuring device 1 detects calculated element data of the transferred sample LB, returns some of the sample LB to the subject M, and discharges the remaining sample into the waste bottle 2 as waste. The measuring device 1 may discharge the entire sample LB to the waste bottle 2 or return the entire sample LB to the subject M. The sample LB discharged into the waste bottle 2 is discarded after being subjected to predetermined processing. A specific configuration of the measuring device 1 will be further described later with a plurality of examples.

The online unit 130 outputs information such as the viscoelasticity of the sample LB calculated by the processing circuitry 160 to an external information system and the like. The display 140 displays information such as the viscoelasticity of the sample LB calculated by the processing circuitry 160. The printer 150 prints information such as the viscoelasticity of the sample LB calculated by the processing circuitry 160.

The processing circuitry 160 includes, for example, a system control function 161, a measurement control function 162, and a calculation function 163. The processing circuitry 160 realizes these functions by a hardware processor executing a program stored in the memory (storage circuit) 170, for example.

The hardware processor may be, for example, circuitry such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). Instead of storing the program in the memory 170, the program may be configured to be directly embedded in the circuit of the hardware processor. In this case, the hardware processor realizes the functions by reading and executing the program embedded in the circuit. The aforementioned program may be stored in the memory 170 in advance, or may be stored in a non-transitory storage medium such as a DVD or a CD-ROM and installed in the memory 170 from the non-transitory storage medium when the non-transitory storage medium is set in a drive device (not shown) of the analysis apparatus 100. The hardware processor is not limited to being configured as a single circuit, and may be configured as one hardware processor by combining a plurality of independent circuits to realize each function. Further, a plurality of components may be integrated into one hardware processor to realize each function.

The system control function 161 acquires information such as a command signal of an operator output from the operation unit 110, analysis conditions, subject information, and measurement items for each sample LB of the subject. The system control function 161 performs control of the entire system, such as control of the measuring device 120 via the measurement control function 162, creation of a calibration table, and control of calculation and output of analysis data on the basis of the acquired information.

The measurement control function 162 controls each member in the measuring device 120 according to instructions from the system control function 161. Control in the measurement control function 162 differs for each of a plurality of measuring devices 120 which will be described below. Therefore, control in the analysis control function will be described in accordance with description of the individual measuring devices 120.

The calculation function 163 acquires each piece of information detected by the measuring device 120. The calculation function 163 calculates the viscoelasticity of the sample LB of the subject on the basis of the acquired information. The calculation function 163 outputs the calculated viscoelasticity information to the online unit 130, the display 140, and the printer 150. The calculation function 163 is an example of a measurer. The calculation function 163 measures at least one of the viscoelasticity or the viscosity of a held blood sample via a mediator in contact with the held blood sample. Further, the calculation function 163 is an example of a calculator. The calculation function 163 calculates an index regarding at least one of the viscoelasticity or the viscosity of the held blood sample on the basis of measurement results.

Before the measuring device 120 detects calculated element data, the analysis apparatus 100 creates a calibration curve using the standard sample LH having known viscoelasticity in the calculation function 163. After the calculation function 163 creates the calibration curve, the measuring device 120 detects calculated element data of the sample LB collected from the subject, and the calculation function 163 calculates the viscoelasticity of the sample LB. The calculation function 163 also evaluates the calculated viscoelasticity. The value calculated by the calculation function 163 may be another index having a correlation with viscoelasticity, such as a dynamic elastic modulus, instead of the value of viscoelasticity itself. The calculation function 163 is an example of a calculator.

The waste (standard sample LH) generated by creating the calibration curve is discharged into the waste bottle 2 and becomes waste, similar to the sample LB that is discarded when the calculation element data is detected. When the amount of waste discharged from the waste bottle 2 exceeds a predetermined amount, the system control function 161 creates waste excess information to that effect, and waste excess information is reported to users via the online unit 130, display 140, or printer 150.

If the standard sample LH remaining in the measuring device 1 or the tube 3 does not affect the subject M, the sample LB used for measurement may be returned to the subject M. An example of returning the sample LB to the subject will be described later. Further, an anticoagulant may be added to the sample LB acquired from the subject M and the effect of the sample LB on the anticoagulant may be measured. Although heparin may be conceived as the anticoagulant, other anticoagulants may be used. An anticoagulant neutralizer, such as heparinase, may be added to the sample containing the anticoagulant. Other anticoagulant neutralizers may be used.

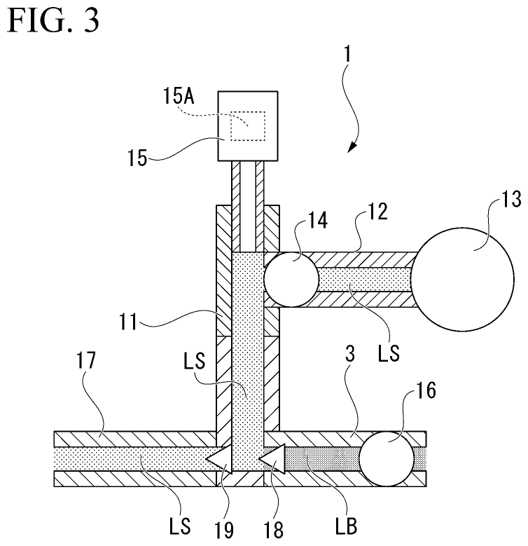

Next, the measuring device 1 in the measuring device 120 of the first embodiment will be described. FIG. 3 is a diagram showing an example of the measuring device 1 of the first embodiment. The measuring device 1 of the first embodiment includes, for example, an attached flow channel 11, a supply pipe 12, a pump 13, a first valve 14, a syringe 15, a second valve 16, a waste pipe 17, a first check valve 18, and a second check valve 19.

A measurement area is formed in the attached flow channel 11. The measurement area is formed as a closed space by being closed by the first valve 14 and the second valve 16. The measurement area is an example of a closed area. The measurement area is filled with a sample LB, a liquid mediator LS such as physiological saline, and the like. FIG. 3 shows a state in which there has been filling with the mediator LS. The mediator may be water, or something other than water or physiological saline, particularly if the sample LB is not returned to the subject M, other than water or physiological saline. The attached flow channel 11 is removable from the measuring device 1. The attached flow channel 11 is an example of a holder. The attached flow channel 11 holds a blood sample collected from subject M.

The supply pipe 12 is connected to the upper end of the attached flow channel 11. The supply pipe 12 supplies the mediator LS to the measurement area. The pump 13 and the first valve 14 are provided in the supply pipe 12. The pump 13 and the first valve 14 are connected to the processing circuitry 160. The pump 13 operates or stops according to control of the measurement control function 162 in the processing circuitry 160. The first valve 14 opens or closes according to control of the measurement control function 162.

The supply pipe 12 communicates with the measurement area when the first valve 14 opens the supply pipe and is shut off from the measurement area when the first valve 14 closes. The pump 13 operates in a state in which the first valve 14 opens to supply the mediator LS to the measurement area through the supply pipe 12. The pump 13 is an example of a supply mechanism or an operating mechanism. The operating mechanism sucks the mediator from the holder and/or discharges the mediator to the holder. The calculation function 163 (measurer) measures at least one of the viscoelasticity or the viscosity of the held blood sample by measuring a response of pressure within the holder to the operation of the operating mechanism.

The syringe 15 is connected to the upper end of the measurement area. By operating the syringe 15, the sample LB flows into the measurement area or is discharged from the measurement area, as will be described later. The syringe 15 is provided with a pressure monitor 15A. The pressure monitor 15A detects the pressure within the measurement area according to the operation of the syringe 15. The pressure monitor 15A generates pressure data based on the detected pressure. The pressure monitor 15A transmits the generated pressure data to processing circuitry 160.

The second valve 16 is provided on the tube 3. The second valve 16 opens and closes according to control of the measurement control function 162. When the second valve 16 opens, the sample LB in the tube 3 can flow into the measurement area. When the second valve 16 closes, the sample LB in the tube 3 stays in the tube 3.

In the measurement area in the attached flow channel 11, an inlet to which the tube 3 is connected and an outlet to which the waste pipe 17 is connected are provided. The tube 3 is connected to the inlet and allows the sample LB to flow into the attached flow channel 11. The sample LB flowing through the tube 3 flows into the measurement area through the inlet. The first tube 3A and the second tube 3B in the tube 3 are examples of inflow tubes.

The sample LB and the mediator LS discharged from the measurement area are discharged to the waste pipe 17 through the outlet. The sample LB flows into the measurement area from the inlet and is discharged from the outlet in one way. The waste pipe 17 is connected to the outlet and communicates with the waste bottle 2 to discharge the sample in the measurement area. The waste pipe 17 is an example of an outflow tube. The sample LB and the mediator LS discharged from the measurement area are discharged to the waste bottle 2 through the waste pipe 17. The sample LB supplied from the subject M moves only within the measurement area of the tube 3 and the attached flow channel 11. Both the tube 3 and the waste pipe 17 are removable from the measuring device 1. Some of all of the attached flow channel 11, the tube 3, and the waste pipe 17 may be non-removable from the measuring device 1.

The first check valve 18 is provided between the inlet and the tube 3. The first check valve 18 prevents backflow from the attached flow channel 11 to the tube 3 provided at the inlet. The second check valve 19 is disposed between the outlet and the waste pipe 17. The second check valve 19 prevents backflow from the waste pipe 17 to the attached flow channel 11.

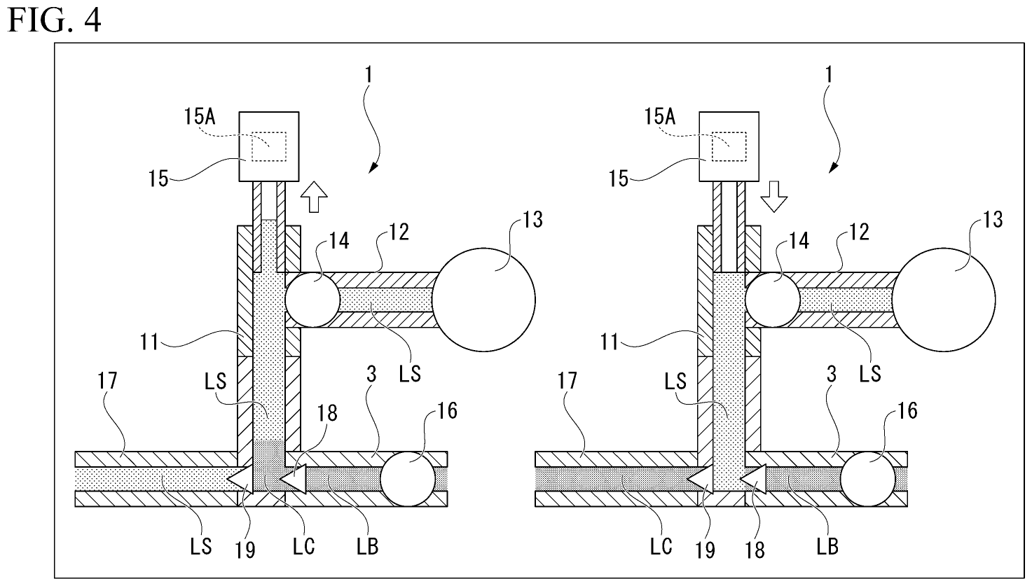

Next, a procedure for calculating the viscoelasticity of the sample LB using the measuring device 1 in the analysis apparatus 100 of the first embodiment will be described with reference to FIGS. 3 and 4. FIG. 4 is a diagram for describing the operation of the measuring device 1 according to the first embodiment at the time of calculating the viscoelasticity of the sample LB. The state shown in FIG. 4 follows the state shown in FIG. 3.

Prior to calculation of the viscoelasticity of the sample LB, the analysis apparatus 100 calculates a pressure detected by the pressure monitor 15A when a standard sample having known viscoelasticity is used and creates a calibration curve. The calibration curve is created, for example, by using a standard sample LH as the sample LB in the procedure for calculating the viscoelasticity of the sample LB which will be described below.

At the time of calculating the viscoelasticity of the sample LB, first, the tube 3 for supplying the sample LB to the measurement area is connected to the inlet of the measurement area in the analysis apparatus 100, as shown in FIG. 3. Subsequently, the measurement control function 162 closes the first valve 14 and sucks the mediator LS in the measurement area with the syringe 15 while the second valve 16 is open, as shown in the left diagram of FIG. 4.

The sample LB flows into the measurement area due to the suction force of the syringe 15 sucking the mediator LS. The pressure monitor 15A detects the pressure within the measurement area when the sample LB flows into the measurement area (hereinafter, inflow pressure). The pressure monitor 15A generates pressure data (hereinafter, first pressure data) based on the detected inflow pressure. The pressure monitor 15A transmits the generated first pressure data to the processing circuitry 160.

Subsequently, as shown in the right diagram of FIG. 4, the measurement control function 162 closes the second valve 16 while keeping the first valve 14 closed, operates the pump 13, and supplies the medium LS to the measurement area to cause the sample LB remaining in the measurement area to be discharged. At this time, the sample LB remaining in the measurement area becomes a waste sample LC. The sample LB used for measurement may flow out to a flow channel through which it is returned to the subject M, and the mediator LS may be discarded to the waste bottle 2.

The pressure monitor 15A detects the pressure within the measurement area when the waste sample LC flows out of the measurement area (hereinafter, outflow pressure). The pressure monitor 15A generates pressure data (hereinafter, second pressure data) based on the detected outflow pressure. The pressure monitor 15A transmits the generated second pressure data to the processing circuitry 160.

In the processing circuitry 160, the calculation function 163 acquires the transmitted first pressure data and second pressure and refers to the pressures indicated by the first pressure data and the second pressure data for the calibration curve created in advance. The calculation function 163 calculates the viscoelasticity of the sample LB on the basis of the result of referring to the inflow pressure and the outflow pressure indicated by the first pressure data and the second pressure data for the calibration curve. The calculation function 163 may calculate the viscoelasticity of the sample LB on the basis of the result of referring to either the inflow pressure or the outflow pressure for the calibration curve.

After the sample LB is caused to flow into the measurement area by the measuring device 1 and the first pressure data and the second pressure data are generated, the first valve 14 and the second valve 16 are closed such that the sample LB is not discharged, and then the attached flow channel 11 is removed. The inlet and the outlet in the measuring device 1 are covered, for example, to prevent communication between the measurement area and the outside air.

The calculation function 163 may calculate the viscosity of the sample LB by referring to either the inflow pressure or the outflow pressure for the calibration curve created in advance. The measurement control function 162 may report the viscoelasticity of the sample LB calculated by the calculation function 163 to the user through the online unit 130, the display 140, or the printer 150. The analysis apparatus 100 may acquire the viscoelasticity of the sample LB at predetermined intervals by repeating the above-described procedure.

For example, the measurement control function 162 may set a predetermined range for the viscoelasticity of the sample LB. In this case, when the viscoelasticity calculated by the calculation function 163 falls outside the predetermined range, the measurement control function 162 may report the fact to the user through the online unit 130, the display 140, or the printer 150.

Alternatively, the measurement control function 162 may calculate the amount of change in the viscoelasticity of the sample LB using the viscoelasticity of the sample LB calculated by the calculation function 163 and determine whether the value of the viscoelasticity falls outside the range within a predetermined time from the amount of change in the viscoelasticity. In this case, if the measurement control function 162 determines that the value of the viscoelasticity value falls outside the range within a predetermined time from the amount of change in the viscoelasticity, the measurement control function 162 may report the fact to the user through the online unit 130, the display 140, or the printer 150. Here, the range of viscoelasticity and the time used for determination may be stored in advance by the analysis apparatus 100, or may be set by the user.

In the analysis apparatus 100 of the first embodiment, the measuring device 1 calculates the viscoelasticity of the sample LB on the basis of the pressure when the sample LB is sucked or discharged using the syringe 15 in the measurement area. Therefore, the viscoelasticity of the sample LB can be calculated in a state in which the sample LB has flowed into the measuring device 1, and thus the viscoelasticity of the sample LB can be measured in real time.

In addition, the sample LB supplied from the subject M moves only within the flow channels of the tube 3 and the attached flow channel 11. Therefore, the used sample LB can be returned to the subject M, and the measuring device 1 can be prevented from being contaminated.

### Second Embodiment

Next, a second embodiment will be described. The analysis apparatus 100 of the second embodiment mainly differs from that of the first embodiment with respect to the configuration of the measuring device 1. In the measuring device 1 of the second embodiment, the supply pipe 12 is not connected to the measurement area, and the pump 13 and the first valve 14 are not provided. In addition, air is interposed between the pressure monitor 15A provided on the syringe 15 through the measurement area and a sample in the measurement area. The pressure monitor 15A and the sample in the measurement area may be in direct contact. In this case, the sample may come into contact with the syringe 15 (the pressure monitor 15A) and become contaminated, and thus the syringe 15 (the pressure monitor 15A) needs to be cleaned.

In the measuring device 1 of the first embodiment, the sample LB is sucked and discharged in a state in which the mediator LS is filled in the measurement area. On the other hand, in the second embodiment, the sample LB is sucked or discharged in a state in which the measurement area is filled with a gas without introduction of a liquid into the measurement area.

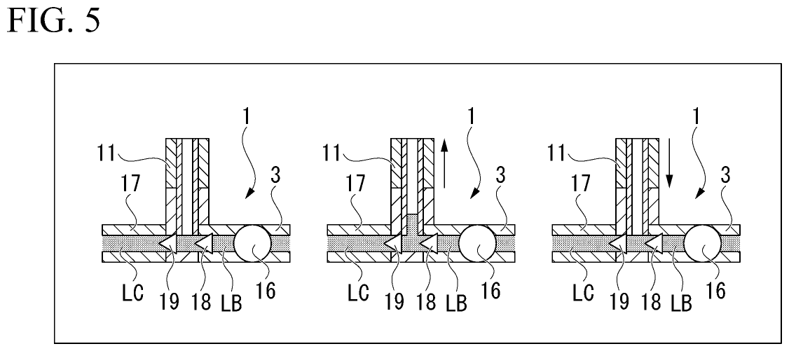

Next, a procedure for calculating the viscoelasticity of the sample LB using the measuring device 1 in the analysis apparatus 100 of the second embodiment will be described with reference to FIGS. 1 to 3 and 5. FIG. 5 is a diagram for describing the operation of the measuring device 1 of the second embodiment at the time of calculating the viscoelasticity of the sample LB. In the second embodiment, prior to calculation of the viscoelasticity of the sample LB, the pressure detected by the pressure monitor 15A when a standard sample LH having known viscoelasticity is used is calculated and a calibration curve is created.

Subsequently, at the time of calculating the viscoelasticity of the sample LB, first, the tube 3 for supplying the sample LB to the measurement area is connected to the inlet of the measurement area in the analysis apparatus 100, as shown in the left diagram of FIG. 5. Here, the sample LB supplied through the tube 3 does not flow into the measurement area and directly flows into the waste pipe 17 because the syringe 15 is not operating.

Subsequently, the measurement control function 162 sucks the inside of the measurement area with the syringe 15 in a state in which the second valve 16 is open. Due to the suction force of the syringe 15 sucking the air in the measurement area, the sample LB flows into the measurement area as shown in the middle diagram of FIG. 5. The pressure monitor 15A detects the inflow pressure when the sample LB flows into the measurement area. The pressure monitor 15A generates first pressure data based on the detected inflow pressure and transmits the first pressure data to the processing circuitry 160.

Subsequently, the measurement control function 162 closes the second valve 16 and applies pressure to the measurement area through the syringe 15, as shown in the right diagram of FIG. 5. Within the measurement area, the pressure applied by the syringe 15 causes the waste sample LC to flow out to the waste pipe 17 through a discharge port. The sample used for measurement may flow out to the flow channel through which it is returned to the subject M.

The pressure monitor 15A detects the outflow pressure when the sample LB (waste sample LC) flows out of the measurement area. The pressure monitor 15A generates second pressure data based on the detected outflow pressure and transmits the second pressure data to the processing circuitry 160.

In the processing circuitry 160, the calculation function 163 acquires the transmitted first pressure data and second pressure, and refers to the pressures indicated by the first pressure data and the second pressure data for the calibration curve created in advance. The calculation function 163 calculates the viscoelasticity of the sample LB on the basis of the result of referring to the inflow pressure and the outflow pressure indicated by the first pressure data and the second pressure data for the calibration curve.

The analysis apparatus 100 of the second embodiment has the same effects as those of the analysis apparatus 100 of the first embodiment. Furthermore, in the analysis apparatus 100 of the second embodiment, the measurement area is not filled with the mediator LS. Therefore, it is possible to improve the measurement sensitivity at the time of measuring the viscosity of the sample LB.

### Third Embodiment

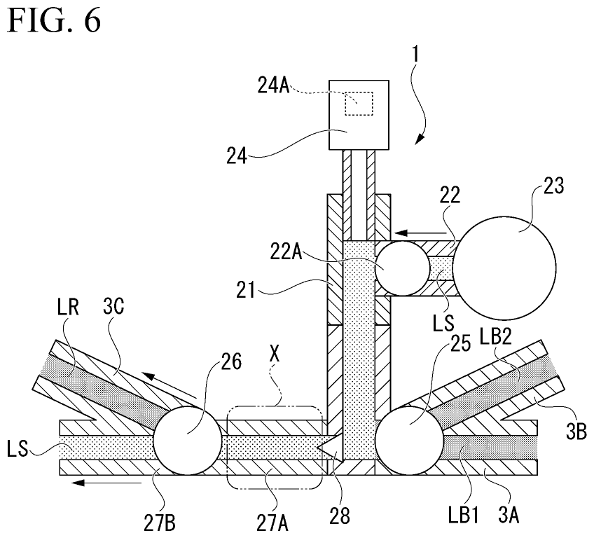

Next, a third embodiment will be described. An analysis apparatus 100 of the third embodiment mainly differs from that of the first embodiment with respect to the configuration of the measuring device 1. FIG. 6 is a diagram showing an example of the measuring device 1 of the third embodiment. The measuring device 1 of the third embodiment includes, for example, an attached flow channel 21, a supply pipe 22, a pump 23, a syringe 24, a first valve 25, a second valve 26, a connection pipe 27A, a waste pipe 27B, a check valve 28, and a return tube 3C. Among these, the attached flow channel 21, the supply pipe 22, the pump 23, and the syringe 24 have the same configurations as the attached flow channel 11, the supply pipe 12, the pump 13, and the syringe 15 of the first embodiment. In the measuring device 1 of the third embodiment, the supply pipe 22 is provided with a valve 22A.

The first valve 25 is a three-way valve provided between the first tube 3A, the second tube 3B, and the inlet of the measurement area. The first tube 3A forms a flow channel through which the first sample LB1 or the first standard sample LH1 flows. The second tube 3B forms a flow channel through which the second sample LB2 or the second standard sample LH2 flows. The first valve 25 switches the flow channel connected to the inlet of the measurement area between the first tube 3A and the second tube 3B on the basis of control of the measurement control function 162.

The second valve 26 is a three-way valve provided between the connection pipe 27A, the waste pipe 27B, and the return tube 3C. The second valve 26 switches the flow channel communicating with the connection pipe 27A between the waste pipe 27B and the return tube 3C on the basis of control of the measurement control function 162. The connection pipe 27A is connected to the outlet of the measurement area. The connection pipe 27A communicates the discharge port of the measurement area and the second valve 26.

The waste pipe 27B is connected to the second valve 26 and communicates with the atmosphere inside the waste bottle 2. The return tube 3C communicates with the subject M. Some or all of the first standard sample LH1 supplied through the first tube 3A, the second standard sample LH2 supplied through the second tube 3B, and the mediator LS filling the measurement area are discharged to the waste bottle 2 via the waste pipe 27B. The first sample LB1 supplied through the first tube 3A and the second sample LB2 supplied through the second tube 3B are returned to the subject M through the return tube 3C. The check valve 28 is provided between the outlet and the connection pipe 27A. The check valve 28 prevents backflow from the connection pipe 27A to the attached flow channel 21.

Next, a procedure for calculating the viscoelasticity of the sample LB using the measuring device 1 in the analysis apparatus 100 of the third embodiment will be described with reference to FIGS. 1, 2, and 6. In the third embodiment, prior to calculation of the viscoelasticity of the sample LB, the pressure detected by the pressure monitor 24A when the standard sample LH having known viscoelasticity is used is calculated and a calibration curve is created. Here, calibration curves when an anticoagulant mixed with the standard sample LH has a high concentration and when it has a low concentration are created using the first standard sample LH1 and the second standard sample LH2.

At the time of creating a calibration curve, the measurement control function 162 controls the first valve 25 and the second valve 26 to connect the first tube 3A through which the first standard sample LH1 flows or the second tube 3B through which the second standard sample LH2 flows to the inlet of the measurement area and to connect the waste pipe 27B to the connection pipe 27A. In this manner, the standard sample LH is caused to flow into the measurement area from the inlet of the measurement area and to be discharged from the outlet.

When an area X in the connection pipe 27A is filled with the standard sample LH, the first valve 25 and the second valve 26 are closed, and the mediator LS in the measurement are sucked by the syringe 24. The pressure monitor 24A detects the pressure when the mediator LS is sucked by the syringe 24 in a state in which the area X is filled with the standard sample LH, generates pressure data, and transmits the pressure data to the processing circuitry 160. The calculation function 163 creates a calibration curve on the basis of the pressure detected by the pressure monitor 24A.

After the pressure monitor 24A finishes detection of the pressure, the connection pipe 27A is communicated with the waste pipe 27B by the second valve 26, the pump 13 is operated to supply the mediator LS to the measurement area, and the standard sample LH remaining in the area X is discarded to the waste bottle 2. Accordingly, a calibration curve is created.

Next, the viscoelasticity of the sample LB is calculated. At the time of calculating the viscoelasticity of the sample LB, the first tube 3A through which the first sample LB1 flows or the second tube 3B through which the second sample LB2 flows is connected to the inlet of the measurement area, and the return tube 3C is connected to the connection pipe 27A. In this manner, the sample LB is caused to flow into the measurement area from the inlet of the measurement area and to be discharged from the outlet, and is caused to be returned to the subject M through the return tube 3C.

When the area X in the connection pipe 27A is filled with the sample LB, the first valve 25 and the second valve 26 are closed, and the mediator LS in the measurement area is sucked by the syringe 24. The pressure monitor 24A detects the pressure when the mediator LS is sucked by the syringe 24 in a state in which the area X is filled with the standard sample LH, generates pressure data, and transmits the pressure data to the processing circuitry 160. The calculation function 163 calculates the viscoelasticity of the sample LB by referring to the pressure detected by the pressure monitor 24A for the calibration curve.

The analysis apparatus 100 of the third embodiment has the same effects as those of the analysis apparatus 100 of the first embodiment. Furthermore, in the analysis apparatus 100 of the third embodiment, the sample used for pressure detection at the time of calculating the viscoelasticity is returned to the subject M. Accordingly, the sample LB can be reliably returned to the subject M. Therefore, it is possible to easily measure the viscoelasticity of the sample in real time.

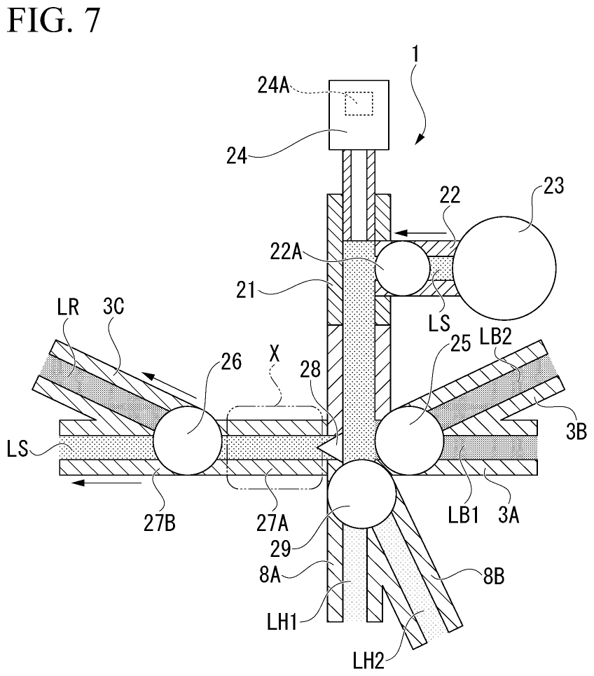

FIG. 7 is a diagram showing a modified example of the measuring device 1 of the third embodiment. In the measuring device 1 of the third embodiment shown in FIG. 6, the first sample LB1 and the first standard sample LH1 are caused to individually flow through the first tube 3A, and the second sample LB2 and the second standard sample LH2 are caused to individually flow through the second tube 3B. Here, if the influence of the standard sample on the subject M is small, for example, the sample LB and the standard sample LH may be caused to flow through a common flow channel, but if the influence of the standard sample on the subject M is large, it is better to divide a common flow channel for the sample LB and the standard sample LH.

The measuring device 1 shown in FIG. 7 differs from the measuring device 1 shown in FIG. 6 in that a third tube 8A, a fourth tube 8B, and a third valve 29 are connected to the measuring device. The first standard sample LH1 flows through the third tube 8A, and the second standard sample LH2 flows through the fourth tube 8B.

The third valve 29 is a three-way valve provided between the third tube 8A, the fourth tube 8B, and the inlet of the measurement area. The third valve 29 switches the flow channel communicating with the inlet of the measurement area between the third tube 8A and the fourth tube 8B on the basis of control of the measurement control function 162. The first standard sample LH1 flows through the third tube 8A, and the second standard sample LH2 flows through the fourth tube 8B.

In the measuring device 1 of the modified example, for example, pressure data is detected in a state in which the area X is filled with the standard sample LH to create a calibration curve. Thereafter, the standard sample LH remaining in the flow channel of the area X is discharged, the flow channel is cleaned by circulating a mediator for example, and then the pressure is detected in a state in which the area X is filled with the sample LB. Accordingly, it is possible to make it difficult for the standard sample to mix with the sample LB at the time of returning the sample LB.

In the measuring device 1 of the modified example, to the measurement area, the first sample LB1 and the second sample LB2 are supplied from the first tube 3A and the second tube 3B and the first standard sample LH1 and the second standard sample LH2 are supplied from the third tube 8A and the fourth tube 8B. Accordingly, the flow channel for the sample of the subject M and the flow channel for the standard sample can be separated, and thus the standard sample can be prevented from being mixed into the subject M.

Although the calculation function 163 in the analysis apparatus 100 calculates viscoelasticity in each of the above-described embodiments, the calculation function 163 may calculate viscosity instead of viscoelasticity and may calculate indexes regarding viscosity, for example, a viscosity coefficient, a coefficient of viscosity, kinematic viscosity, and the like in addition to viscosity itself. The calculation function 163 may calculate some or all of viscoelasticity and viscosity or some or all of indexes regarding viscoelasticity and viscosity.

### Fourth Embodiment

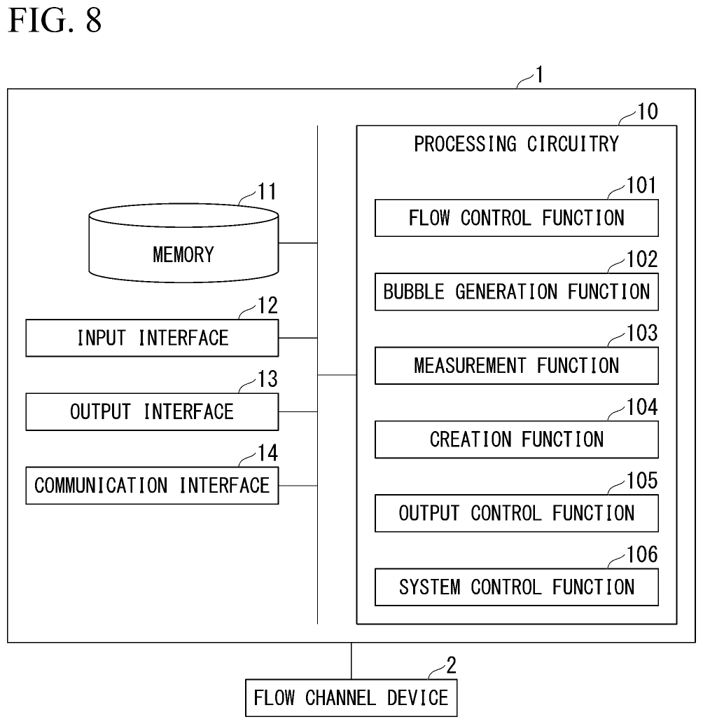

An analysis system according to a fourth embodiment will be described with reference to the block diagram of FIG. 8.

The analysis system includes an analysis apparatus 1 and a flow channel device 2. The analysis apparatus 1 includes processing circuitry 10, a memory 11, an input interface 12, an output interface 13, and a communication interface 14.

The processing circuitry 10 includes a flow control function 101, a bubble generation function 102, a measurement function 103, a creation function 104, an output control function 105, and a system control function 106.

The flow control function 101 controls inflow of a target sample and a mediator into a measurement area within the flow channel device 2. Here, the target sample is assumed to be blood, whole blood, plasma, or the like, but any substance may be used as the target sample as long as it is a liquid for which an index of at least one of viscosity or viscoelasticity of the target sample is to be measured. The mediator is assumed to be water or physiological saline, but may also be Ringer's solution, oil, or the like. In addition, an example of a case in which viscoelasticity is measured as an index will be described below.

The bubble generation function 102 heats the liquid within the measurement area to generate bubbles. The term “liquid” as used herein refers to a mediator or a liquid in which a mediator and a target sample are mixed.

The measurement function 103 measures the viscoelasticity of the target sample on the basis of the geometrical characteristics (size, etc.) of the bubbles and/or a response due to generation of the bubbles (outflow amount of the target sample from the measurement area). The measurement function 103 is an example of a measurer. The measurement function 103 measures at least one of the viscoelasticity or the viscosity of a held blood sample via a mediator in contact with the held blood sample. Further, the measurement function 103 is an example of a calculator. The measurement function 103 calculates an index regarding at least one of the viscoelasticity or the viscosity of the held blood sample on the basis of the measurement results.

The creation function 104 creates a calibration curve on the basis of quantitative values measured for a standard sample having known viscoelasticity and known viscoelasticity values.

The output control function 105 outputs the viscoelasticity measurement results of the target sample to the outside.

The system control function 106 performs general control regarding viscoelasticity measurement processing.

The memory 11 is a storage device such as a read only memory (ROM), a random access memory (RAM), a hard disk drive (HDD), a solid state drive (SSD), and an integrated circuit storage device for storing various types of information. Further, the memory 11 may be a drive device or the like that reads/writes various types of information from/to portable storage media such as a CD-ROM drive, a DVD drive, and a flash memory. The memory 11 does not necessarily need to be realized by a single storage device. For example, the memory 11 may be realized by a plurality of storage devices. Alternatively, the memory 11 may be in another computer connected to the analysis apparatus 1 via a network.

The memory 11 stores a processing program and the like according to the present embodiment. This program may be stored in advance in the memory 11, for example. Alternatively, the program may be stored in a non-transitory storage medium, distributed, read from the non-transitory storage medium, and installed in the memory 11, for example.

The input interface 12 receives various input operations from the user, converts the received input operations into electrical signals, and outputs the electrical signals to the processing circuitry 10. The input interface 12 according to the present embodiment is connected to input equipment such as a mouse, a keyboard, a trackball, a switch, a button, a joystick, a touchpad, and a touch panel to which instructions are input by touching an operation surface. Further, the input equipment connected to the input interface 12 may be input equipment provided in another computer connected via a network or the like.

The output interface 13 outputs data generated by the analysis apparatus 1, such as viscoelasticity measurement results, to output equipment such as a display, a printer, a projector, and a speaker. When data is output from a speaker, the data is converted into an audio signal and output. Further, the output equipment may be mounted on the analysis apparatus 1, or may be disposed outside the analysis apparatus 1 and connected in a wired or wireless manner.

The communication interface 14 performs data communication with medical information management applications, hospital information systems, radiology department information systems, and the like.

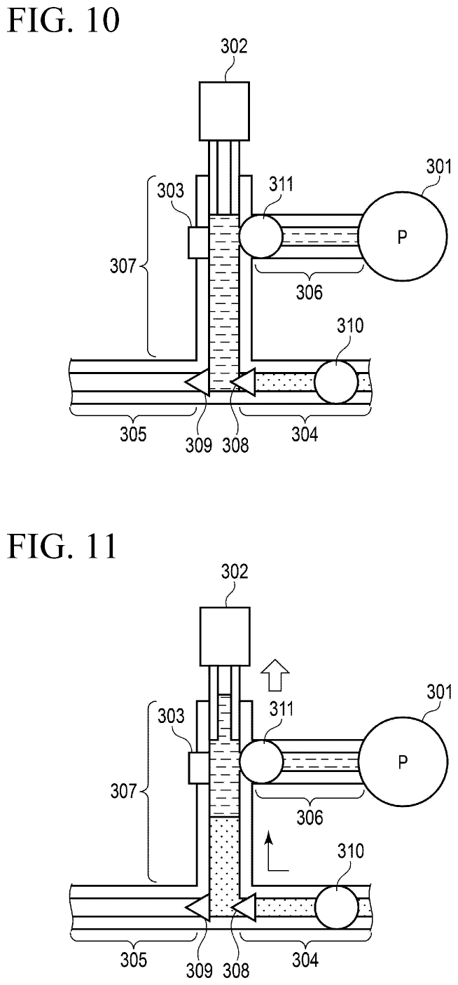

The flow channel device 2 is a flow channel having a measurement area, in which the viscoelasticity of a target sample is determined according to flow of the target sample and a mediator into the measurement area. Details of the flow channel device 2 will be described later with reference to FIG. 10 and the following figures.

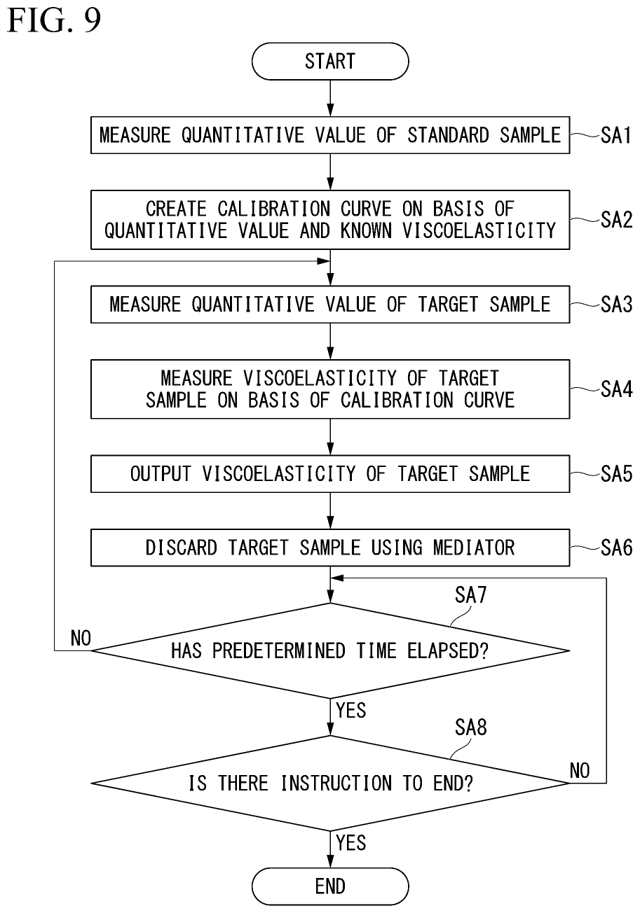

Next, an example of analysis processing of the analysis system according to the present embodiment will be described with reference to the flowchart of FIG. 9.

In step SA1, the processing circuitry 10 measures a quantitative value correlated with the viscoelasticity of a standard sample having a known viscoelasticity value using the flow channel device 2 before measuring the viscoelasticity of a sample. Specifically, viscoelasticity measurement according to a bubble generation method is assumed here, and the processing circuitry 10 causes a mediator and the standard sample to flow into a measurement area through the flow control function 101. The processing circuitry 10 generates bubbles within the measurement area through the bubble generation function 102. The processing circuitry 10 measures sizes of the bubbles or an outflow amount of the standard sample within the measurement area through the measurement function 103. That is, the bubble sizes or the outflow amount of the standard sample are used as quantitative values. The bubble sizes may be obtained, for example, by capturing an image of the bubbles with a camera and measuring the sizes of the bubbles from the image. The outflow amount may be measured using a graduated container, a volumetric camera, or the like. The processing circuitry 10 measures the viscoelasticity of two or more standard samples having different viscoelasticities through the above-described measurement method using the measurement function 103.

In step SA2, the processing circuitry 10 creates a calibration curve on the basis of the measured values measured in step SA1 for each of the two or more standard samples and known viscoelasticity values through the creation function 104.

In step SA3, the processing circuitry 10 measures quantitative values of a target sample. As a method of measuring the viscoelasticity of the target sample, the same method as in step SA1 may be used. That is, the processing circuitry 10 causes the mediator and the target sample to flow into the measurement area through the flow control function 101. The processing circuitry 10 generates bubbles within the measurement area through the bubble generation function 102. The processing circuitry 10 measures sizes of the bubbles or an outflow amount of the target sample within the measurement area through the measurement function 103.

In step SA4, the processing circuitry 10 measures the viscoelasticity of the target sample using the calibration curve of the standard sample through the measurement function 103. For example, when the corresponding relationship between the bubble sizes of the standard sample and the viscoelasticity of the standard sample is generated in advance as a calibration curve, the viscoelasticity value corresponding to the bubble sizes of the liquid in the measurement area may be calculated as the viscoelasticity of the target sample on the basis of the calibration curve.

In step SA5, the processing circuitry 10 outputs measurement results of the target sample through the output control function 105. For example, the value of the viscoelasticity of the target sample may be output as a measurement result to an external display connected to the analysis apparatus 1 or to a display if the display is included in the analysis apparatus 1. Alternatively, the viscoelasticity of the target sample may be printed on a paper medium and output from a printer connected to the analysis apparatus 1. Alternatively, information on the viscoelasticity of the target sample may be output by voice from a speaker connected to the analysis apparatus 1.

In step SA6, the processing circuitry 10 causes the mediator to flow into the measurement area and discard the measured target sample through the flow control function 101.

In step SA7, the processing circuitry 10 determines whether or not a predetermined period has elapsed since the immediately previous viscoelasticity measurement timing through the system control function 106. If the predetermined period has elapsed, processing returns to step SA3 and the same processing is repeated. If the predetermined period has not elapsed, processing proceeds to step SA8.

In step SA8, the processing circuitry 10 determines whether or not there is an instruction to end target sample analysis processing through the system control function 106. For example, if the user gives an instruction to end measurement, analysis processing ends. If analysis processing is does not end, processing returns to step SA7 and the same processing is repeated.

Creation of the calibration curve for the standard sample in steps SA1 and SA2 may be performed each time the target sample is measured. Once the calibration curve is created, the same calibration curve may be used during subsequent measurement of the viscoelasticity of a target sample.

Furthermore, if the measured viscoelasticity value of the target sample falls outside a predetermined range (outside an allowable range of viscoelasticity) in step SA4, the processing circuitry 10 may notify the user of the information through the measurement function 103. For example, the processing circuitry 10 may cause the display to display an alert indicating that the viscoelasticity value falls outside the predetermined range through the output control function 105. In addition, if the processing circuitry 10 determines that the viscoelasticity value falls outside the predetermined range within a predetermined period from time-series change in the viscoelasticity value obtained by measuring the viscoelasticity value of the target sample in time series for each predetermined period, that is, the amount of change in viscoelasticity through the measurement function 103, the processing circuitry 10 may notify the user of the information. For example, the processing circuitry 10 may calculate the slope of time-series data of the viscoelasticity value as the amount of change in viscoelasticity, and determine whether or not the viscoelasticity value falls outside the predetermined range within the predetermined period on the basis of the slope through the measurement function 103. If it is determined that the viscoelasticity value falls outside the predetermined range within the predetermined period, the processing circuitry 10 may cause the display to display, for example, an alert through the output control function 105.

Next, a structural example of the flow channel device 2 and a viscoelasticity measurement method using the flow channel device 2 will be described with reference to FIGS. 10 to 12. FIG. 10 shows a first structural example of the flow channel device 2.

The flow channel device 2 includes a pump 301, a syringe 302, a bubble generation mechanism 303, a sample inflow channel 304, an outflow channel 305, a mediator inflow channel 306, a measurement flow channel 307, and a first check valve 308, a second check valve 309, a first on-off valve 310, and a second on-off valve 311.

The pump 301 is connected to the mediator inflow channel 306, sucks up a mediator from a container (not shown) in which the mediator is stored, and causes the mediator to flow into the mediator inflow channel 306. It is assumed that the pump 301 according to the present embodiment controls inflow of the mediator according to the flow control function 101. The syringe 302 is connected to the measurement flow channel 307, and sucks and discharges a liquid in the measurement flow channel 307.

The bubble generation mechanism 303 heats the liquid in the measurement flow channel 307 to generate bubbles. In the bubble generation mechanism 303, for example, a thin film heater is disposed in the measurement flow channel 307, and when the heater is energized, film boiling occurs in the liquid in contact with the heater, generating bubbles. When blood is directly heated, thermal denaturation occurs, and thus the bubble generation mechanism 303 may be disposed, for example, at a position facing the mediator inflow channel 306, which will be described later and/or may be disposed on the side of the syringe 302 such that only the mediator in the measurement area can be heated.

The sample inflow channel 304 is a flow channel that is connected to the measurement flow channel 307 and allows a target sample to flow into the measurement flow channel 307.

The outflow channel 305 is a channel that is connected to the measurement flow channel 307 and allows the target sample to flow out from the measurement flow channel 307.

The mediator inflow channel 306 is a channel that is connected to the measurement flow channel 307 and allows the mediator to flow into the measurement flow channel 307.

The measurement flow channel 307 is connected to the sample inflow channel 304, the outflow channel 305, and the mediator inflow channel 306, and is a closed area in which liquid bubbles are formed. The measurement flow channel 307 is an example of a holder. The measurement flow channel 307 holds a blood sample collected from a subject.

The first check valve 308 is disposed such that the target sample flows only from the sample inflow channel 304 to the measurement flow channel 307. That is, backflow from the measurement flow channel 307 to the sample inflow channel 304 is prevented.

The second check valve 309 is disposed such that the target sample flows only from the measurement flow channel 307 to the outflow channel 305. That is, backflow from the outflow channel 305 to the measurement flow channel 307 is prevented.

The first on-off valve 310 is disposed in the sample inflow channel 304 and controls inflow of the target sample by controlling opening and closing. It is assumed that the first on-off valve 310 controls inflow of the target sample through the flow control function 101.

The second on-off valve 311 is disposed in the mediator inflow channel 306 and controls inflow of the mediator by controlling opening and closing. It is assumed that the second on-off valve 311 controls inflow of the mediator through the flow control function 101.

It is assumed that the processing circuitry 10 electronically controls driving of the pump 301 and the syringe 302, opening and closing of the valves of the flow channel device 2, and driving of the bubble generation mechanism 303 through the flow control function 101. The pump 301, the syringe 302, the bubble generation mechanism 303, the first on-off valve 310, and the second on-off valve 311 may use a general electronically controllable mechanism, and thus detailed description thereof will be omitted here.

In addition, the flow channel device 2 may be made of a disposable material for one-time analysis processing. For example, at least one component among the sample inflow channel 304, the outflow channel 305, the mediator inflow channel 306, the measurement flow channel 307, the first check valve 308, the second check valve 309, the first on-off valve 310, and the second on-off valves 311 may be made of thermoplastic resin such as polyvinyl chloride or synthetic resin such as silicon. Accordingly, it is possible to prevent contamination by replacing the channel device for each measurement processing of a target sample.

Next, a first process of the viscoelasticity measurement method according to the first structural example is shown in FIG. 11.

In FIG. 11, a state in which the inside of the measurement flow channel 307 is filled with a mediator is assumed to be an initial state. In the initial state, the processing circuitry 10 opens the first on-off valve 310 and close the second on-off valve 311 through the flow control function 101. The processing circuitry 10 drives the syringe 302 through the flow control function 101 such that the syringe 302 sucks up the mediator in the measurement flow channel 307 to the side of the syringe 302 by performing a suction operation, and thus the liquid amount of target sample corresponding to the volume sucked by the syringe 302 flows into the measurement flow channel 307.

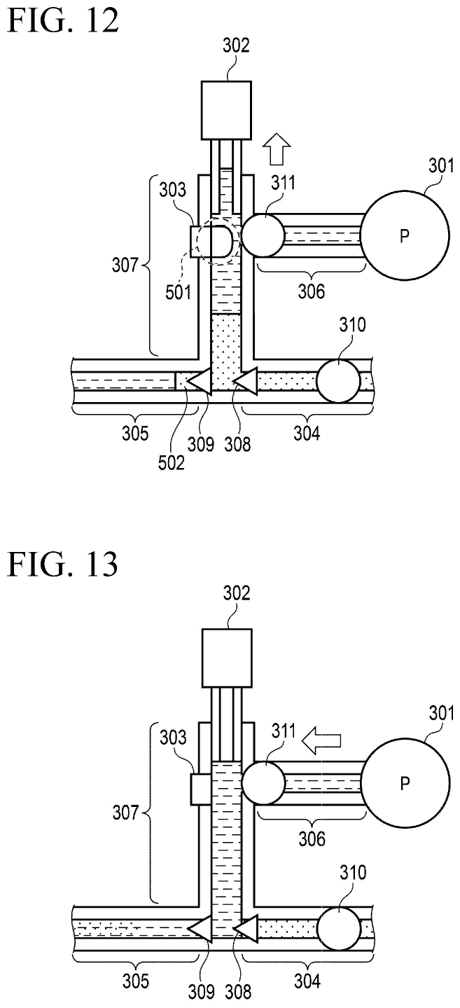

Next, a second process of the viscoelasticity measurement method according to the first structural example of the flow channel device 2 is shown in FIG. 12.

In FIG. 12, the processing circuitry 10 closes both the first on-off valve 310 and the second on-off valve 311 through the flow control function 101. The processing circuitry 10 drives the bubble generation mechanism 303 to generate bubbles by heating the liquid in the measurement flow channel 307 through the bubble generation function 102. The processing circuitry 10 measures sizes of the generated bubbles or the volume of the liquid that has flowed out of the outflow channel 305 through the measurement function 103. The bubble sizes depend on the viscoelasticity of the liquid. Specifically, the greater the viscoelasticity of the target sample, the smaller the bubble sizes, and the smaller the viscoelasticity of the target sample, the greater the bubble sizes. Therefore, as shown in FIG. 9, by generating the corresponding relationship between the bubble sizes of the standard sample and the viscoelasticity of the standard sample as a calibration curve in advance, a viscoelasticity value on the calibration curve corresponding to the measured bubble sizes of the liquid can be calculated as the viscoelasticity of the target sample.

In addition to the bubble sizes, the viscoelasticity may be measured from the relationship between the above-described viscoelasticity and the volume of the target sample flowing out from the outflow channel 305. That is, when the viscoelasticity of the target sample is high, the bubble sizes of the liquid decrease, and thus the volume (liquid amount) of the target sample extruded from the measurement flow channel 307 to the outflow channel 305 decreases. On the other hand, when the viscoelasticity of the target sample is low, the bubble sizes of the liquid increase, and thus the volume (liquid amount) of the target sample extruded from the measurement flow channel 307 to the outflow channel 305 increases. Therefore, by generating the corresponding relationship between the volume in which the standard sample is extruded and the viscoelasticity of the standard sample as a calibration curve in advance according to the above relationship, a viscoelasticity value on the calibration curve corresponding to the volume of the target sample extruded from the measurement flow channel 307 to the outflow channel 305 can be calculated as the viscoelasticity of the target sample.

Next, a third process of the viscoelasticity measurement method according to the first structural example of the flow channel device 2 is shown in FIG. 13.

When viscoelasticity measurement of the target sample ends through the second process described above, the processing circuitry 10 closes the first on-off valve 310 and opens the second on-off valve 311 through the flow control function 101. The processing circuitry 10 operates the pump 301 to cause the mediator to flow into the measurement flow channel 307 to push the liquid in the measurement flow channel 307 out to the outflow channel 305 through the flow control function 101.

As a result, the liquid in the measurement flow channel 307 can flow to be discarded to prepare for the next viscoelasticity measurement. In addition, since the target sample moves only within the flow channel device 2, specifically, only between the sample inflow channel 304, the outflow channel, 305 and the measurement flow channel 307, viscoelasticity measurements can be performed continuously without contaminating other components of the analysis apparatus.

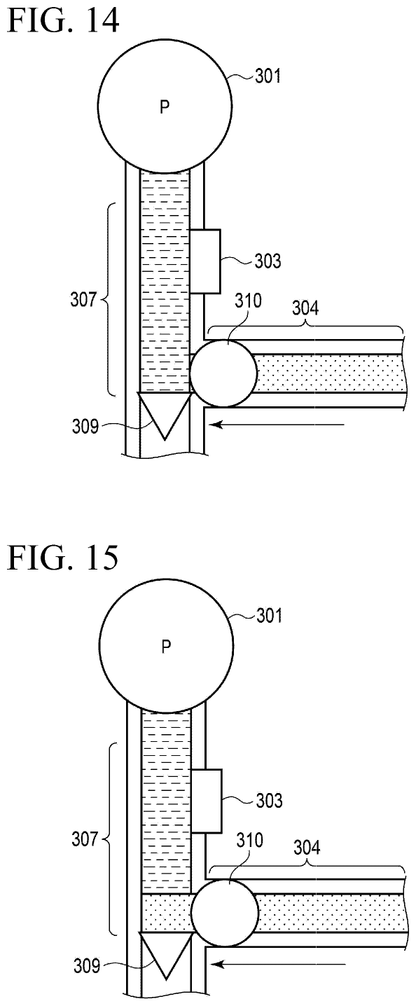

Next, a second structural example of the flow channel device 2 according to the fourth embodiment is shown in FIG. 14.

The second structural example of the flow channel device 2 shows a case in which the syringe 302 is not disposed in the measurement flow channel 307 as compared with the first structural example. The flow channel device 2 according to the second structural example includes the pump 301, the bubble generation mechanism 303, the sample inflow channel 304, the outflow channel 305, the measurement flow channel 307, and the first on-off valve 310.

Although FIG. 14 shows an example in which the first check valve 308 is not provided, the first check valve 308 and the second check valve 309 may be disposed in the same manner as in the first structural example.

Next, a first process of the viscoelasticity measurement method according to the second structural example is shown in FIG. 15.

In FIG. 15, the processing circuitry 10 opens the first on-off valve 310 of the sample inflow channel 304 to allow the target sample to flow into the measurement flow channel 307 through the flow control function 101. A small amount of the target sample flows into the measurement flow channel 307 as compared to a case in which a syringe causes the target sample to flow.

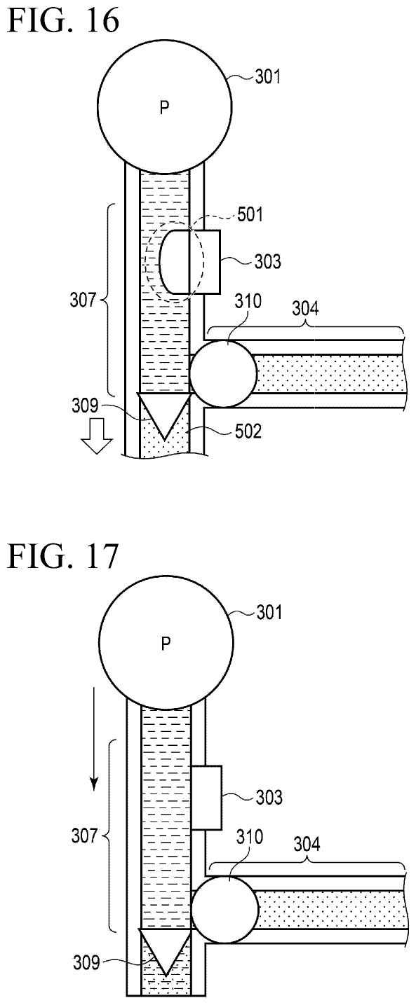

Next, a second process of the viscoelasticity measurement method according to the second structural example is shown in FIG. 16.

The processing circuitry 10 closes the first on-off valve 310 through the flow control function 101. The processing circuitry 10 drives the bubble generation mechanism 303 to generate bubbles by heating the liquid in the measurement flow channel 307 through the bubble generation function 102. The processing circuitry 10 measures the sizes of the generated bubbles or the volume of the liquid flowing out from the outflow channel 305 through the measurement function 103 as in the case of the first structural example of the flow channel device 2.

Next, a third process of the viscoelasticity measurement method according to the second structural example is shown in FIG. 17.

When viscoelasticity measurement of the target sample ends, the processing circuitry 10 closes the first on-off valve 310, opens the second on-off valve 311, and causes the mediator to flow into the measurement flow channel 307 by the pump 301 through the flow control function 101. Accordingly, the sample can be discharged from the closed area of the measurement flow channel 307.

Also according to the second structural example of the flow channel device 2, the target sample moves only between the sample inflow channel 304, the outflow channel 305, and the measurement flow channel 307, and thus it is possible to perform viscoelasticity measurement without contaminating other components of the analysis system including the analysis apparatus 1.

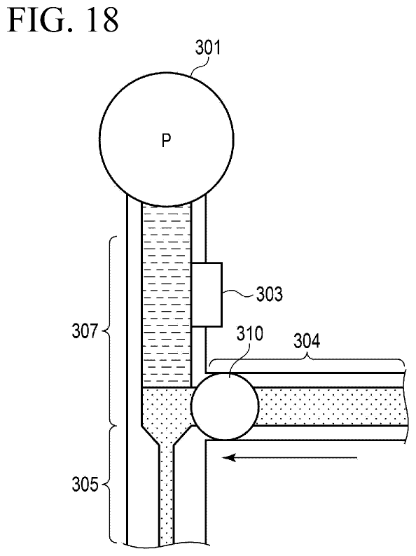

Next, a modified example of the second structural example of the flow channel device 2 is shown in FIG. 18.

In the modified example of the second structural example, the flow passage of the outflow channel 305 is designed to be narrower or longer in order to greatly affect the target sample on bubbles generated in the measurement flow channel 307. FIG. 18 shows an example in which the outflow channel 305 in the second structural example of the flow channel device 2 is designed to be narrow and long. Although not shown, the second check valve 309 may be disposed in the outflow channel 305.

In the second structural example of the flow channel device 2 or the modified example of the second structural example described above, the target sample may be caused to flow into the measurement flow channel 307 according to contraction of bubbles. This is because, for example, when bubbles disappear in a state in which the first on-off valve 310 is open, the liquid amount of the target sample corresponding to the volume of the bubbles is sucked up into the measurement flow channel 307. Therefore, the processing circuitry 10 can cause a larger amount of target sample to flow into the measurement flow channel 307 than that in a case where the first on-off valve 310 is open such that the target sample flows into the measurement flow channel 307 by generating bubbles and making the bubbles disappear in a state in which the first on-off valve 310 is open through the bubble generation function 102.

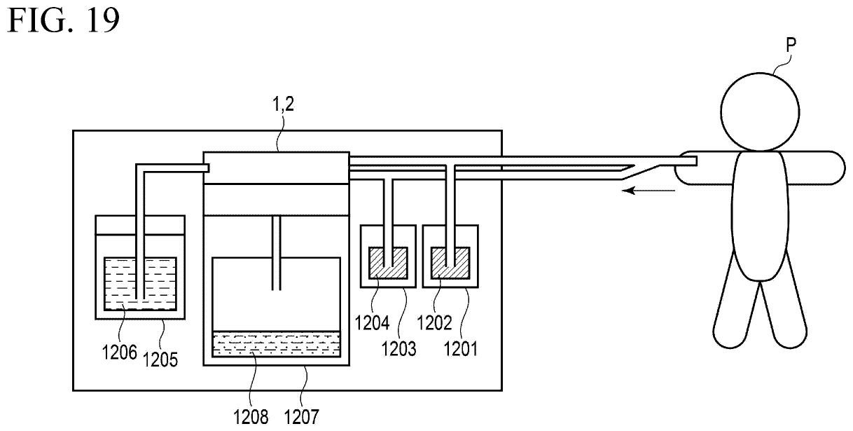

Next, a conceptual diagram showing a usage example of the analysis system according to the fourth embodiment is shown in FIG. 19.

Here, an analysis system including a patient P and the analysis apparatus 1 is shown. Blood collected from the patient P is used as a target sample, the target sample flows into the flow channel device 2 through a blood collection tube, and the viscoelasticity of the target sample is measured.

The analysis system shown in FIG. 19 includes the analysis apparatus 1, the flow channel device 2, a first standard sample container 1201, a second standard sample container 1203, a mediator container 1205, and a waste container 1207.

In the flow channel device 2, the sample inflow channel 304 is connected to the blood collection tube from the patient. The mediator inflow channel 306 is connected such that a mediator 1206 in the mediator container 1205 can be sucked up by the pump 301. The outflow channel 305 is connected such that the liquid containing the measurement sample that has been measured can be discharged to the waste container 1207.

The first standard sample container 1201 is a container for storing a first standard sample 1202 for creating a calibration curve.

The second standard sample container 1203 is a container for storing a second standard sample 1204 for creating a calibration curve. The first standard sample 1202 and the second standard sample 1204 have different viscoelasticities, and the values of the viscoelasticities are known.

The mediator container 1205 stores the mediator 1206.

The waste container 1207 is a container that allows the liquid discharged from the outflow channel 305 to be able to be discarded without coming into contact with the outside air or human hands.

Each flow channel (the sample inflow channel 304, the outflow channel 305, the mediator inflow channel 306, and measurement flow channel 307) is controlled such that it has a predetermined temperature (constant temperature control).

As a preliminary step of measuring the target sample of the patient P, a calibration curve is created through the above-described viscoelasticity measurement method using the first standard sample 1202 and the second standard sample 1204 (steps SA1 and SA2 in FIG. 9).

Thereafter, viscoelasticity measurement of the target sample described above is performed (steps SA3 to SA8 in FIG. 9). Each time the target sample of the patient P is measured, the first on-off valve 310 and the second on-off valve 311 of the flow channel device 2 are closed, and the measured liquid is discarded from the outflow channel 305 to the waste container 1207. A mounting port on the side of the analysis apparatus 1 is covered with a cover to prevent communication with the outside air.

According to the fourth embodiment described above, a target sample such as patient's blood is caused to flow into the measurement area using the flow channel device that generates bubbles in the measurement flow channel, and the sizes of the bubbles or the amount of outflow from the measurement area due to generation of bubbles are measured. The viscoelasticity of the target sample is measured on the basis of the bubble sizes or the amount of outflow and a calibration curve.

Accordingly, it is possible to obtain the viscoelasticity value of the target sample within a short time according to comparison with the calibration curve. In addition, it is possible to prevent contamination and infection of the analysis apparatus and medical staffs by forming the flow channel device of a disposable material. That is, it is possible to improve convenience in medical practice such as surgery and diagnosis.

### Fifth Embodiment

In a fifth embodiment, it is assumed that the viscoelasticity of a target sample is measured using an anticoagulant.

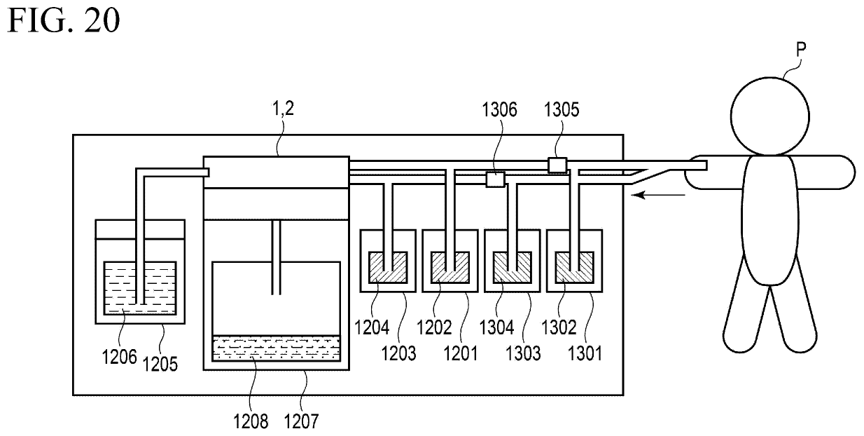

FIG. 20 shows a usage example of an analysis system according to the fifth embodiment.

For example, it is assumed that the viscoelasticity of the blood of a patient during surgery and transfused blood is to be measured in real time, a target sample is obtained by adding an anticoagulant (heparin, sodium citrate, or the like) to the blood obtained from the patient, and the influence of the anticoagulant on the target sample is measured.

The analysis system shown in FIG. 20 includes a first mixed medication container 1301 and a second mixed medication container 1303 in addition to the analysis apparatus 1, the flow channel device 2, the first standard sample container 1201, the second standard sample container 1203, the mediator container 1205, and the waste container 1207 shown in FIG. 19.

The first mixed medication container 1301 is a container that stores a first anticoagulant 1302.

The second mixed medication container 1303 is a container that stores a second anticoagulant 1304. It is assumed that the second anticoagulant 1304 is a different type of medication from the first anticoagulant 1302 and does not have the same anticoagulability as that of the first anticoagulant 1302, that is, has low or high anticoagulability.

The analysis system adds two different anticoagulants to the blood obtained from the patient and performs viscoelasticity measurements on the two mixed samples. Mixing units 1305 and 1306 for mixing the target sample and the anticoagulants may be disposed in the blood collection tube.

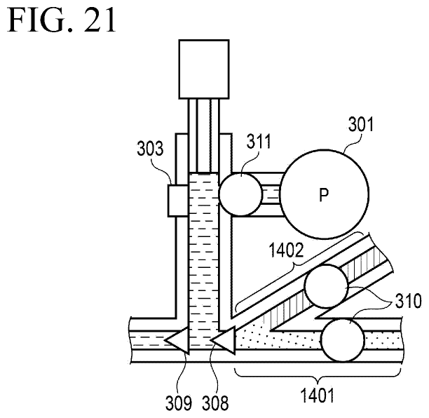

Next, an example of a configuration of the flow channel device 2 according to the fifth embodiment is shown in FIG. 21.

The flow channel device 2 shown in FIG. 21 differs from the configuration of the flow channel device 2 shown in FIG. 10 in that it includes a first sample inflow channel 1401 into which the target sample to which the first anticoagulant 1302 has been added flows and a second sample inflow channel 1402 into which the target sample alone flows without an anticoagulant added thereto. The second anticoagulant 1304 may flow into the second sample inflow channel 1402.

Although a case in which the first on-off valve 310 is provided in each of the first sample inflow channel 1401 and the second sample inflow channel 1402 is assumed, a valve that switches the flow channel into the measurement flow channel 307 between the first sample inflow channel 1401 and the second sample inflow channel 1402 may be configured.

Since there are two target samples to be measured, measurement of the viscoelasticity of the target sample inflowing from the first sample inflow channel 1401 and measurement of the viscoelasticity of the target sample inflowing from the second sample inflow channel 1402 are alternately performed. Accordingly, when no anticoagulant is added to the second sample inflow channel 1402, for example, the processing circuitry 10 can calculate the amount of anticoagulant that results in a predetermined viscoelasticity, that is, a predetermined anticoagulability through the measurement function 103 by comparing the viscoelasticity of the anticoagulant-mixed sample inflowing from the first sample inflow channel 1401 with the viscoelasticity of the sample without an anticoagulant inflowing from the second sample inflow channel 1402.

In cardiac surgery, and the like, if a patient has already been injected with an anticoagulant such as heparin, a neutralizing agent for the anticoagulant which has a heparin neutralizing effect may be administered and the degree of influence (coagulability) of the neutralizing agent on the anticoagulant-mixed sample may be measured. Examples of neutralizing agents may include heparinase and protamine sulfate. Specifically, a first neutralizing agent is stored in the first mixed medication container 1301, a second neutralizing agent is stored in the second mixed medication container 1303, and the first neutralizing agent and the second neutralizing agent are added to an anticoagulant-mixed sample obtained from the patient to generate two mixed samples. Thereafter, viscoelasticity measurement may be performed on the mixed samples in the same manner.

According to the fifth embodiment described above, similarly to the fourth embodiment, the viscoelasticity value of the target sample can be obtained within a short time according to comparison with a calibration curve. Furthermore, an appropriate amount of anticoagulant to be added to the patient can be ascertained because the influence of the anticoagulant on the target sample or the influence of the neutralizing agent on the anticoagulant-mixed sample.

Meanwhile, the term “processor” used in the above description means, for example, a circuit such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)). When the processor is, for example, a CPU, the processor realizes the functions thereof by reading and executing a program stored in a storage circuit. On the other hand, if the processor is, for example, an ASIC, the program is not stored in the storage circuit, and the functions are directly embedded as a logic circuit in the circuit of the processor. Each processor of the present embodiment is not limited to being configured as a single circuit for each processor, and may be configured as a single processor by combining a plurality of independent circuits to realize the functions thereof. Furthermore, a plurality of components in the figures may be integrated into a single processor to realize the functions thereof.

In addition, each function according to the embodiments can also be realized by installing a program for executing the above-described processing on a computer such as a workstation and deploying the program on a memory. In this case, the program that allows the computer to execute the above-described method can be distributed by being stored in a storage medium such as a magnetic disk (a hard disk, etc.), an optical disk (a CD-ROM, a DVD, etc.), a semiconductor memory, or the like.

According to at least one embodiment described above, it is possible to obtain measurement results within a short time by including a holder that holds a blood sample collected from a subject, and a measurer that measures at least one of the viscoelasticity or the viscosity of the held blood sample via a mediator in contact with the held blood sample.

### Appendix 1

An analysis apparatus including:
a measuring device including a detector that detects a pressure in a closed area in a flow channel through which a sample flows; and
a calculator that calculates an index regarding at least one of viscoelasticity or viscosity of the sample on the basis of the pressure in the closed area.

### Appendix 2

The measuring device may further include a syringe for sucking the sample in the closed area, and the pressure in the closed area may include a pressure according to the operation of the syringe.

### Appendix 3

An inlet that allows the sample to flow into the closed area, and an outlet through which the sample flows out from the closed area may be further included.

### Appendix 4

The sample may be caused to flow into the closed area from the inlet and to flow out from the outlet in one way.

### Appendix 5

A supply mechanism for supplying a liquid mediator to the closed area may be further included.

### Appendix 6

The mediator may be supplied by the supply mechanism to the closed area filled with the sample to cause the sample within the closed area to be discharged.

### Appendix 7

The closed area is formed through an attached flow channel,
an inflow tube that is connected to the inlet and allows the sample to flow into the attached flow channel, and an outflow tube that is connected to the outlet and allows the sample within the closed area to be discharged, and
at least one of the attached flow channel, the inflow tube, or the outflow tube may be removable.

### Appendix 8

Air may be interposed between the detector and the sample via the closed area.

### Appendix 9

The sample removed from a subject is caused to flow into the closed area, and the sample discharged from the closed area may be returned to the subject.

### Appendix 10

An anticoagulant may be mixed with the sample.

### Appendix 11

The anticoagulant may have any one of a plurality of concentrations.

### Appendix 12

An analysis method including:
detecting a pressure in a closed area in a flow channel through which a sample flows according to operation of a syringe that sucks or discharges the sample in the closed area; and
evaluating an index regarding at least one of viscoelasticity or viscosity of the sample on the basis of the pressure in the closed area.

### Appendix 13

An analysis apparatus including:
a controller that controls inflow of a target sample and a mediator into a measurement area;
a generator that heats a liquid in the measurement area to generate bubbles; and
a measurer that measures an index of at least one of viscosity or viscoelasticity of the target sample on the basis of sizes of the bubbles or an outflow amount of the target sample from the measurement area due to generation of the bubbles.

### Appendix 14

The index of the target sample may be measured using a flow channel device formed by a measurement flow channel that forms the measurement area, a first inflow channel connected to the measurement flow channel and allowing the target sample to flow into the measurement area, a second inflow channel connected to the measurement flow channel and allowing the mediator to flow into the measurement area, and an outflow channel through which the target sample and the mediator flow out from the measurement flow channel.

### Appendix 15

The flow channel device may further include:
a first check valve that prevents inflow of a liquid from the measurement flow channel to the first inflow channel; and
a second check valve that prevents inflow of a liquid from the outflow channel to the measurement flow channel.

### Appendix 16

The sample in the measurement area may be discharged by flowing the mediator into the measurement flow channel from the second inflow channel after the index is measured.

### Appendix 17

At least one of the measurement flow channel, the first inflow channel, the second inflow channel, or the outflow channel may be disposable.

### Appendix 18

The target sample may come into contact only within the flow channel device.

### Appendix 19

An output that outputs a result of measurement of the index may be further included.

### Appendix 20

If a value of the index falls outside a predetermined range, the measurer may notify a user of the information.

### Appendix 21

If it is determined that the value of the index falls outside the predetermined range within a predetermined period from an amount of change in the index obtained by measuring the index in a time series, the measurer notifies the user of the information.

### Appendix 22

An analysis method including:
controlling inflow of a target sample and a mediator into a measurement area;
heating a liquid in the measurement area to generate bubbles; and
measuring an index of at least one of viscosity or viscoelasticity of the target sample on the basis of sizes of the bubbles or an outflow amount of the target sample from the measurement area due to generation of the bubbles.

### Appendix 23

An analysis program causing a computer to realize:
a control function of controlling inflow of a target sample and a mediator into a measurement area;
a generation function of heating a liquid in the measurement area to generate bubbles; and
a measurement function of measuring an index of at least one of viscosity or viscoelasticity of the target sample on the basis of sizes of the bubbles or an outflow amount of the target sample from the measurement area due to generation of the bubbles.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An analysis apparatus comprising:
a holder configured to hold a blood sample collected from a subject; and
processing circuitry configured to measure at least one of viscoelasticity or viscosity of the held blood sample via a mediator in contact with the held blood sample.

2. The analysis apparatus according to claim 1, wherein the processing circuitry is further configured to calculate an index regarding the at least one of the viscoelasticity or the viscosity of the held blood sample on the basis of results of the measurement.

3. The analysis apparatus according to claim 1, further comprising an operating mechanism configured to suck and/or discharge the mediator from and/or to the holder,
wherein the processing circuitry is further configured to measure the at least one of the viscoelasticity or the viscosity of the held blood sample by measuring a response of a pressure within the holder to operation of the operating mechanism.

4. The analysis apparatus according to claim 1, further comprising a generator configured to generate bubbles by heating the mediator,
wherein the processing circuitry is further configured to measure the at least one of the viscoelasticity or the viscosity of the held blood sample by measuring geometric characteristics of the bubbles and/or a response due to generation of the bubbles.

5. The analysis apparatus according to claim 4, wherein the processing circuitry is further configured to measure the at least one of the viscoelasticity or the viscosity of the held blood sample on the basis of sizes of the bubbles or an inflow amount of the blood sample from the holder due to generation of the bubbles.

6. The analysis apparatus according to claim 1, wherein the holder includes a measurement flow channel that forms a measurement area, and
the measurement flow channel is provided with an inlet through which the blood sample flows into the measurement flow channel, and an outlet through which the blood sample flows out from the measurement flow channel.

7. The analysis apparatus according to claim 6, further comprising:
an inflow tube that is connected to the inlet and allows the blood sample to flow into the measurement flow channel; and
an outflow tube that is connected to the outlet and allows the blood sample within the measurement flow channel to be discharged,
wherein at least one of the measurement flow channel, the inflow tube, or the outflow tube is removable.

8. An analysis method, using a computer of an analysis apparatus including a holder configured to hold a blood sample collected from a subject, comprising measuring at least one of viscoelasticity or viscosity of the held blood sample via a mediator in contact with the held blood sample.

9. A computer-readable non-transitory storage medium storing a program causing a computer of an analysis apparatus including a holder configured to hold a blood sample collected from a subject to measure at least one of viscoelasticity or viscosity of the held blood sample via a mediator in contact with the held blood sample.
